(19) United States
(12) Patent Application Publication
(10) Pub. No.: US 2003/0091556 A1
(43) Pub. Date: May 15, 2003

(54) Methods of inhibiting tumor growth and angiogenesis with anastellin

(76) Inventors: Erkki I. Ruoslahti, Rancho Santa Fe, CA (US); Ming Yi, La Jolla, CA (US)

Correspondence Address: CAMPBELL & FLORES LLP, 7th Floor, 4370 La Jolla Village Drive, San Diego, CA, 92122, US

(21) Appl. No.: 10/005,171

(22) Filed: Dec. 3, 2001

Related U.S. Application Data
(60) Provisional application No. 60/331,357, filed on Dec. 4, 2000.

(51) Int. Cl. A61K 38/48; C12N 9/64

(52) U.S. Cl. 424/94.64; 435/226

(57) ABSTRACT

The invention provides a substantially pure composition of anastellin (SEQ ID NO: 1) and fibrinogen as well as a substantially pure composition of superfibronectin and superfibrinogen. The invention also provides methods of inhibiting angiogenesis, tumor growth and metastasis by administering anastellin (SEQ ID NO: 1) alone or in combination with fibrinogen.

This application claims the benefit of U.S. Provisional Application No. 60/______ , (yet to be assigned) filed Dec. 4, 2000, which was converted from U.S. Ser. No. 09/729,657, and is incorporated herein by reference.

This invention was made with government support under grant number CA74238 and the Cancer Center Support Grant CA30199 awarded by the National Cancer Institute and grant DAMD17-00-1-0556 awarded by the Department of Defense. The United States Government has certain rights in this invention.

### BACKGROUND OF THE INVENTION

This invention relates generally to the field of cancer biology and, more specifically to compositions and methods for inhibiting angiogenesis, tumor growth, and metastasis.

This year about 552,200 Americans are expected to die of cancer, an average of more than 1,500 people per day. Cancer is the second leading cause of death in the United States, where one out of every four deaths is due to cancer. Since 1990, approximately 13 million new cases have been diagnosed and nearly five million lives have been lost to cancer. In 2000, about 1,220,100 new cancer cases will be diagnosed. While progress in preventing and treating cancer has been made, including particular success against Hodgkin's lymphoma and certain other forms, many types of cancer remain substantially impervious to prevailing treatment protocols.

One of the hallmarks of cancer as well as that of over seventy other diseases, including diabetic blindness, age-related macular degeneration, rheumatoid arthritis and psoriasis, is the body's loss of control over angiogenesis. Angiogenesis-dependent diseases result when new blood vessels either grow excessively or insufficiently. Excessive angiogenesis occurs when diseased cells produce and release abnormal amounts of angiogenic growth factors, overwhelming the effects of natural angiogenesis inhibitors. The resulting new blood vessels feed diseased tissues, which in turn destroy normal tissues.

Upon their release, angiogenic growth factors diffuse into nearby tissues and bind to specific receptors located on the endothelial cells of nearby preexisting blood vessels. Once growth factors bind to their receptors, the endothelial cells become activated and send signals from the cell surface to the nucleus. As a result, the endothelial cell's machinery begins to produce new molecules including enzymes that create tiny holes in the basement membrane that surrounds existing blood vessels. As the endothelial cells begin to proliferate, they migrate out through the enzyme-created holes of the existing blood vessel towards the diseased tissue; in the case of cancer, the endothelial cells migrate towards the tumor. Specialized molecules called adhesion molecules or integrins provide anchors that allow the new blood vessel to sprout forward. Additional enzymes, among them matrix metalloproteinases (MMPs), are produced to dissolve the tissue in front of the growing blood vessel tip to allow for its continued tissue invasion. As the vessel extends, the tissue is remolded around the vessel and endothelial cells roll up to form a new blood vessel. Subsequently, individual blood vessels connect to form blood vessel loops that can circulate blood. Finally, the newly formed blood vessels are stabilized by specialized muscle cells (smooth muscle cells, pericytes) that provide structural support and blood flow through the neovascularized tissue begins.

Significantly, angiogenesis is one of the critical events required for cancer metastasis. Metastasis, the ability of cancer cells to penetrate into lymphatic and blood vessels, circulate through the bloodstream, and invade and grow in normal tissues elsewhere makes cancer a life-threatening disease. Tumor angiogenesis is the proliferation of a network of blood vessels that penetrates into cancerous growths, supplying nutrients and oxygen and removing waste products.

Anti-angiogenic therapies, aimed at halting new blood vessel growth, are needed to treat cancer as well as other conditions characterized by excessive angiogenesis. In the case of cancer, there exists a particular need to supplement existing methods of treating cancer with anti-angiogenic therapies aimed at halting angiogenesis, tumor growth and metastasis. The present invention satisfies this need and provides related advantages as well.

### SUMMARY OF THE INVENTION

The invention provides a substantially pure composition of anastellin (SEQ ID NO: 1) and fibrinogen as well as a substantially pure composition of superfibronectin and superfibrinogen. The invention also provides methods of inhibiting angiogenesis, tumor growth and metastasis by administering anastellin (SEQ ID NO: 1) alone or in combination with fibrinogen.

### BRIEF DESCRIPTION OF THE DRAWINGS

FIG. 1 shows the effect of systematic treatment with anastellin (SEQ ID NO: 1) on the growth of human xenograft tumors in mice.

FIG. 2 shows polymerization of fibrinogen by anastellin (SEQ ID NO: 1).

FIG. 3 shows inhibition of KRIB osteosarcoma growth by anastellin (SEQ ID NO: 1), sFN and sFBG.

FIG. 4 shows inhibition of MDA-MD-435 breast carcinoma growth by anastellin (SEQ ID NO: 1), sFN and sFBG.

FIG. 5 shows immunohistochemical staining for blood vessels in sections of KRIB tumors. Representative microscopic fields are shown from the tumors in the vehicle alone group (A), the anastellin (B), sFN (C), and sFBG (D) groups. Magnification 400×; scale bar 50 pm.

FIG. 6 shows quantification of decreased blood vessel density in MDA-MB-435 tumors from mice treated with anastellin, sFN or sFBG. The graph depicts the mean and SEM for the number of blood vessels in the tumors from the six mice in each treatment group shown in FIG. 4.

### DETAILED DESCRIPTION OF THE INVENTION

This invention provides substantially pure compositions containing anastellin (SEQ ID NO: 1) and fibrinogen. The invention also provides substantially pure compositions containing superfibrinogen (sFBG) and superfibronectin (sFN). Anastellin (SEQ ID NO: 1). Anastellin (SEQ ID NO: 1) and the compositions of the invention are useful for inhibition of tumor growth, angiogenesis and metastasis.

The invention is further directed to methods of inhibiting tumor growth, angiogenesis and metastasis through administration of anastellin (SEQ ID NO: 1) alone, as well as through administration of compositions containing anastellin (SEQ ID NO: 1) and fibrinogen.

As used herein, the term “anastellin” refers to an amino acid fragment of the first type III fibronectin repeat that is about 76 amino acids in length and designated herein as SEQ ID NO: 1. The anastellin peptide spans residues 600 to 674 of fibronectin according to the numbering of Kornblihtt et al., EMBO J. 4(7):1755-9 (1985), which is incorporated herein by reference, and has the following sequence: NAPQPSHISK YILRWRPKNS VGRWKEATIP GHLNSYTIKG LKPGVVYEGQ LISIQQYGHQ EVTRFDFTTT STSTP (SEQ ID NO:1). Thus, anastellin can be 76 amino acids in length and have the identical amino acid sequence that is set forth as SEQ ID NO: 1. Functionally, anastellin is an inhibitor of tumor growth, tumor angiogenesis and metastasis as shown herein. Anastellin also functions as a fibronectin polymerizing agent and a fibrinogen polymerizing agent.

Anastellin (SEQ ID NO: 1) is representative of a growing class of anti-angiogenic substances that can derived from extracellular matrix and blood proteins by proteolysis or other modifications well known in the art. These anti-angiogenic substances include angiostatin (O'Reilly et al., Cell 79:315-328 (1994)), endostatin (O'Reilly et al., Cell 88: 277-285 (1997)), heparin-binding fragments of fibronectin (Homandberg et al., Am. J. Path. 120:327-332 (1985); Biochim. Biophys. Acta 874:61-71 (1986)) and a modified form of antithrombin (O'Reilly et al., Science 285:1926-1928 (1999)).

While the mechanism of activity of anti-angiogenic substances is unknown, the teachings regarding anastellin (SEQ ID NO: 1) provided by the present invention elucidate a possible general mechanism of action for anti-angiogenic substances. Angiogenic substances bind to one or more adhesion proteins: angiostatin and its parent protein plasminogen can bind vitronectin (Kost et al., Eur.J. Biochem. 236:682-688 (1996)), endostatin has been shown to bind to fibulins and nidogen-2 (Miosge et al., FASEB J. 13:1743-1750 (1999)), and the anti-angiogenic form of antithrombin is similar to the modified antithrombin that binds to vitronectin (Ill and Ruoslahti, J. Biol. Chem. 260:15610-15615 (1985)). Anastellin (SEQ ID NO: 1) not only binds to fibronectin and fibrinogen in serum, it also polymerizes these proteins in vitro and in vivo. Fibronectin, fibrinogen and each of the other ligands for the various anti-angiogenic substances described above, are adhesion proteins containing the RGD cell adhesion sequence as described by Ruoslahti, Ann. Rev. Cell Dev. Biol. 12:697-715 (1996), which is incorporated herein by reference. Moreover, these anti-angiogenic substances bind to the αvβ3 integrin, which is expressed at high levels in angiogenic endothelial cells and plays an important role in angiogenesis as described by Brooks et al., Science 264:569-571 (1994), which is incorporated herein by reference. Therefore, it is likely that anti-angiogenic substances polymerize an RGD-containing protein in vivo, followed by binding of the polymers to the αvβ3 integrin on angiogenic endothelial cells, which leads to inhibition of cell proliferation and causes apoptosis.

Fibronectin is a prototypic extracellular matrix (ECM) protein that is deposited by various types of cells into an adhesive fibrillar meshwork of protein as described in Hynes, Fibronectins, Springer-verlag, New York (1990). Fibronectin, and the extracellular matrix in general, control many cellular functions, including growth, migration, differentiation, tissue repair, embryogenesis and survival. The signals that control these behaviors are transmitted from the extracellular matrix to the cell by integrins, a family of transmembrane receptors as reviewed by Clark and Brugge, Science 268:233-239 (1995) and Giancotti and Ruoslahti, Science 285:1028-1032 (1999), both of which are incorporated herein by reference. Malignant cells often by-pass the ECM-integrin signaling system, gaining independence from the adhesive constraints of the exracellular matrix and the requirement of extracellular matrix contact for survival as described by Frisch and Ruoslahti, Curr. Opi. Cell Biol. 9:701-706 (1997), which is incorporated herein by reference.

Fibronectin exists in two main forms: as an insoluble glycoprotein dimer that serves as a linker in the ECM and as a soluble disulphide linked dimer found in the plasma (plasma FN). While the plasma form is synthesized by hepatocytes, the ECM form is made by fibroblasts, chondrocytes, endothelial cells, macrophages, as well as certain epithelial cells. Sequences of fibronectin are known to those skilled in the art, for example, as in Kornblihtt, et al., EMBO J. 4:1755-1759 (1985), incorporated herein by reference, or, are available from Genbank (Accession Numbers 2506872 and 31396), NCBI, NIH, and easily searchable, for example, on the internet. NCBI's database, which is known to those skilled in the art, is herein incorporated by reference in its entirety.

As used herein, the term “superfibronectin” or “sFN” refers to multimers of fibronectin of high relative molecular mass, polymeric fibrillar forms of fibronectin and high molecular weight aggregates of fibronectin as described in Morla, A., et al., Nature 367:193-196 (1994), which is incorporated herein by reference. Superfibronectin can be generated in vitro by treating purified fibronectin or fragments of fibronectin in solution with a fibronectin polymerizing agent containing at least one fibronectin-fibronectin binding site as described in Morla et al., supra, 1994, and in U.S. Pat. No. 5,922,676, which is incorporated herein by reference. Fibronectin polymerizing agents are fragments of fibronectin that have the property of enhancing fibronectin-fibronectin binding and fibronectin-fibronectin intermolecular associations. This enhancement of fibronectin-fibronectin binding and/or intermolecular association can generate superfibronectin in vitro and in vivo. A preferred form of superfibronectin useful for the compositions and methods of the invention is prepared by mixing anastellin (SEQ ID NO: 1) and fibronectin as described by Morla et al., supra, 1994. However, this invention is not limited by either the mechanism or the particular fibronectin polymerizing agent utilized to prepare superfibronectin.

Superfibronectin can be a product of aggregation or cross-linking of fibronectin into high molecular weight forms. While the multimers can be formed by treating soluble fibronectin with fibronectin polypeptide fragments, superfibronectin also includes high molecular weight aggregates of fibronectin formed by any other process or mechanism of aggregate formation. Superfibronectin aggregates can form with or without intermolecular covalent crosslinking, or any combination thereof. Superfibronectin or its components can be purified or synthesized from natural, synthetic, or recombinant sources or a combination thereof as well as obtained commercially (Sigma, St. Louis, Mo.).

Superfibronectin, compared to fibronectin, adheres to cells more strongly by a factor of ten, an increase can be measured and quantified, as described in Morla et al., supra, 1994. In addition, cell migration is also measurably reduced by superfibronectin compared to fibronectin and can be quantitated using a wound assay as described in Morla et al., supra, 1994. Furthermore, superfibronectin has anti-metastatic properties as described in Example IV and in Pasqualini et al., Nature Medicine 2: 1197-1203 (1996). As described in Examples II and III, superfibronectin also is an inhibitor of tumor gowth and angiogenesis. Superfibronectin includes modified forms of superfibronectin or its constituent fibronectin fragments so long as they retain biological activity. Useful modifications can be identified using the adhesion assay and wound assay described in Morla et al., supra, 1994, and further using the tumor growth inhibition, metastasis and angiogenesis assays described in Examples II, III and IV below.

Fibrinogen is a plasma glycoprotein composed of six polypeptide chains (α, β, γ)₂held together by disulfide bonds and organized in symmetrical dimeric fashion. In the last stage of blood coagulation, the serine protease thrombin cleaves the α and β chains of fibrinogen, releasing fibrinopeptides A and B, respectively, and converting fibrinogen into fibrin monomers. The spontaneous polymerization of fibrin monomers initiates fibrin clotting with the formation of protofibrils, which subsequently aggregate in a lateral fashion to form fibers that are held together by noncovalent and electrostatic forces. Sequences of fibrinogen are known to those skilled in the art, for example, as in Rixon et al., Biochemistry 22: 3237-3244 (1983); Chung et al., Biochemistry 22: 3244-3250 (1983); and Chung et al., Biochemistry 22: 3250-3256 (1983), incorporated herein by reference, or, are available from Genbank, NCBI, NIH, and easily searchable, for example, on the internet. NCBI's database, which is known to those skilled in the art, is herein incorporated by reference in its entirety.

Following polymerization, the transglutaminase Factor XIIIa, which is produced by the action of thrombin on Factor XIII, catalyzes the formation of covalent bonds between specific glutamine and lysine residues located in the carboxy-terminal regions of adjacent γ chains and between a chains to form γ-γ dimers and α polymers. These intermolecular bonds crosslink the fibrin gel network and, together with Factor XIIIa-mediated crosslinking of α2-antiplasmin to fibrin, solidify the clot.

As used herein, the term “superfibrinogen” or “sFBG” refers to multimers of fibrinogen of high relative molecular mass, polymeric fibrillar forms of fibrinogen and high molecular weight aggregates of fibrinogen. Functionally, compared to fibrinogen, superfibrinogen can inhibit tumor growth, angiogenesis and metastasis. Superfibrinogen includes modified forms of superfibrinogen or its constituent fibrinogen fragments so long as they retain functional activity. Useful modifications can be identified using the tumor growth, angiogenesis and metastasis assays described in Examples II, III and IV.

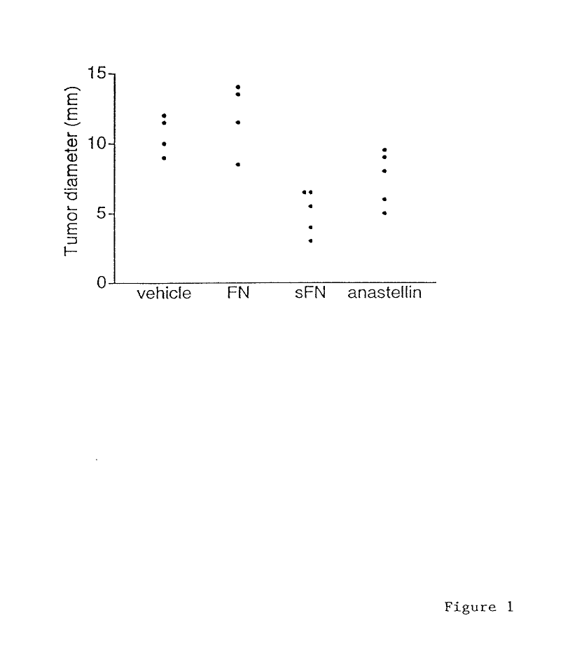

Superfibrinogen can be a product of aggregation or cross-linking of fibrinogen into high molecular weight forms (FIG. 1). While the multimers can be formed by treating soluble fibrinogen with anastellin (SEQ ID NO: 1), as described in Example I below, superfibrinogen encompasses high molecular weight aggregates of fibrinogen formed by any other polymerizing agent as well as any process or mechanism of aggregate formation. Polymerizing agents useful for preparation of superfibrinogen include the transglutaminase Factor XIII as well as any naturally occuring or synthetic transamidating enzyme or other compounds capable of polymerizing fibrinogen. Superfibrinogen aggregates can form with or without intermolecular covalent crosslinking, as well as through any combination thereof. Similarly, superfibrinogen or its components can be purified or synthesized from natural, synthetic, or recombinant sources or a combination thereof.

The invention provides substantially pure compositions of anastellin (SEQ ID NO: 1) and fibrinogen that yield superfibrinogen and anastellin (SEQ ID NO: 1) upon polymerization of the constituent fibrinogen. The substantially pure invention compositions of anastellin (SEQ ID NO: 1) and fibrinogen are useful for inhibiting angiogenesis, tumor growth and metastasis. The invention also provides substantially pure compositions of anastellin (SEQ ID NO: 1), fibronectin and fibrinogen, that yield superfibronectin and superfibrinogen, along with anastellin (SEQ ID NO: 1), upon polymerization of the constituent fibronectin and fibrinogen. Anastellin (SEQ ID NO: 1) and the substantially pure compositions of the invention have substantial therapeutic usefulness as inhibitors of angiogenesis, tumor growth and metastasis as disclosed herein. In addition, the substantially pure compositions of the invention retain the functional activities of the constituent polypeptides as described herein.

As used herein, the term “substantially pure” when used in reference to a composition of the invention is intended to mean a molecule that the composition relatively free from cellular components cellular components or other contaminants that are not the desired composition or its constituent polypeptides.

The invention provides a method of preparing superfibrinogen by contacting fibrinogen with anastellin (SEQ ID NO: 1) under conditions that allow formation of superfibrinogen.

Superfibrinogen can be prepared by mixing fibrinogen and anastellin (SEQ ID NO: 1) or any other fibrinogen polymerizing agent in an appropriate buffer solution such as, for example, phosphate-buffered saline under the conditions described in Example I. Buffer solutions useful for preparing the compositions of the invention are known to those in the art and it is understood that a buffer can be prepared based on the desired ionic strength, pH, molar concentration and optimum temperature range. For example, a buffer appropriate for preparing the compositions of the invention contains 9.0 g NaCl, 0.25 g KCl, 1.43 g Na₂HPO₄ and 0.25 KH₂PO₄ in 1000 cm³ of distilled water, has a pH of 7.2 and a temperature of 37° C.

In addition, physiological buffers useful for in vivo adminstration are well known in the art and further desribed below. The preparation of superfibronectin is known and described in the art (Pasqualini et al., supra, 1996) as well as described in Example I.

Anastellin (SEQ ID NO: 1), fibronectin, fibrinogen, superfibronectin and superfibrinogen are collectively referred to herein as the constituent polypeptides of the invention. The constituent polypeptides of the invention are intended to encompass variants having substantially the same amino acid sequence as the reference constituent polypeptide and exhibit at least one of the functional activities thereof. An anastellin polypeptide of the invention can have the same amino acid sequence set forth in SEQ ID NO: 1. Alternatively, an anastellin polypeptide of the invention can have one or more amino acid alterations compared to the amino acid sequence set forth in SEQ ID NO: 1 that do not significantly change its biological activity. Similarly, the fibronectin and fibrinogen components of superfibronectin and superfibrinogen, respectively, can have either the same amino acid sequences or can have one or more alterations compared to the amino acid sequences set forth herein that do not significantly change the functional activities of superfibronectin and superfibrinogen. In addition, superfibronectin and superfibrinogen can consist of any fibronectin or fibrinogen polymerizing agent, respectively, that does not significantly change the functional activity of superfibronectin and superfibrinogen.

An anastellin polypeptide useful for the compositions and methods of the invention can have substantially the same sequence as SEQ ID NO: 1 and can further be a polypeptide, fragment or segment having an identical amino acid sequence as SEQ ID NO: 1, or a polypeptide, fragment or segment having a similar, non-identical sequence that is considered by those skilled in the art to be a functional equivalent of SEQ ID NO: 1. Furthermore, a fibronectin polypeptide useful for the compositions and methods of the invention can have substantially the same sequence as those known in the art and described in, for example, Kornblihtt, et al., supra, 1985, incorporated herein by reference, and can further be a polypeptide, fragment or segment having an identical amino acid sequence to one known in the art, or a polypeptide, fragment or segment having a similar, non-identical sequence that is considered by those skilled in the art to be a functional equivalent of fibronectin. Similarly, a fibrinogen polypeptide useful for the compositions and methods of the invention can have substantially the same sequence as those known in the art and described in, for example, Rixon et al., supra, (1983); Chung et al., supra, (1983); and Chung et al., supra, (1983) and can further be a polypeptide, fragment or segment having an identical amino acid sequence to one known in the art, or a polypeptide, fragment or segment having a similar, non-identical sequence that is considered by those skilled in the art to be a functional equivalent of fibrinogen. Sequences of fibrinogen are known to those skilled in the art and are available from Genbank (Accession Numbers 182406, 182429 and 182438), NCBI, NIH, and easily searchable, for example, on the internet. A functional equivalent of a constituent polypeptide of the invention retains at least one of the functional activities of its reference peptide. Functional activities of anastellin (SEQ ID NO: 1) include inhibition of tumor growth, tumor angiogenesis and metastasis as described herein as well as the ability to polymerize both fibronectin and fibrinogen in vitro and in vivo. Functional activities of fibronectin include the ability to assemble into superfibronectin. Functional activities of fibrinogen include the ability to assemble into superfibrinogen as well as the ability form blood clots as part of the coagulation cascade of proteins. In addition, functional activities of superfibronectin and superfibrinogen include inhibition of tumor growth, tumor angiogenesis and metastasis as described herein. A functional equivalent of a constituent polypeptide of the invention such as anastellin (SEQ ID NO: 1), fibronectin or fibrinogen includes those amino acid sequences that are sufficient for retention of a particular functional activity associated with the reference polypeptide. For example, a functional equivalent of a constituent polypeptide of the invention can include those amino acid sequences that are sufficient for assembly of superfibronectin or superfibrinogen aggregates. In addition, a functional equivalent of a constituent polypeptide of the invention can include those amino acid sequences sufficient for inhibition of angiogenesis, tumor growth or metastasis.

A constituent polypeptide of the invention can have at least 70%, at least 80%, at least 81%, at least 83%, at least 85%, at least 90%, at least 95% or more identity to the respective sequences of anastellin, fibronectin and fibrinogen set forth as SEQ ID NOS: 1, 2 and 3, respectively. The constituent polypeptides of the invention also encompass modified forms of naturally occurring amino acids such as D-stereoisomers, non-naturally occurring amino acids, amino acid analogues and mimetics so long as such polypeptides retain a functional activity of the reference polypeptide.

The constituent polyeptides of the invention include those polypeptides, fragments or segments having an amino acid sequence identical to that of the constituent polypeptide of the invention, or a polypeptide, fragment or segment having a similar, non-identical sequence that is considered by those skilled in the art to be a functional equivalent of the reference constituent polypeptide of the invention. Such a functional equivalent or functional fragment of a constituent polypeptide of the invention exhibits at least one functional activity of the reference polypeptide and can have, for example, at least 6 contiguous amino acid residues from the reference constituent polypeptide, at least 8, 10, 15, 20, 30 or 40 amino acids, and often has at least 50, 75, 100, 200, 300, 400 or more amino acids of a polypeptide of the invention, up to the full length polypeptide minus one amino acid. The appropriate length and amino acid sequence of a functional fragment of a constituent polypeptide of the invention can be determined by those skilled in the art, depending on the intended use of the functional fragment. For example, a functional fragment of anastellin (SEQ ID NO: 1) is intended to refer to a portion of anastellin that still retains some or all of the fibronectin or fibrinogen polymerizing activity of the reference polypeptide. Therefore, a functional fragment of anastellin (SEQ ID NO: 1) can contain at least one or more binding sites necessary for crosslinking fibronectin or fibrinogen into aggregates. Alternatively, a functional fragment of anastellin (SEQ ID NO: 1) can contain that part of the amino acid sequence of the reference polypeptide required for inhibition of tumor growth, tumor angiogenesis and metastasis as described herein. Similarly, a functional fragment of fibronectin or fibrinogen can contain at least one or more binding sites necessary for aggregation by a polymerizing agent. In addition, a functional fragment of a superfibronectin or superfibrinogen can contain that part of the amino acid sequence of fibronectin or fibrinogen, respectively, required for the inhibition of tumor growth, tumor angiogenesis and metastasis as described herein.

Minor modifications in the primary amino acid sequence of anastellin (SEQ ID NO: 1), fibronectin, superfibronectin, fibrinogen and superfibrinogen can result in polypeptides that retain substantially equivalent function. These modifications can be deliberate, as through site-directed mutagenesis, or can be accidental such as through spontaneous mutation. For example, it is understood that only a portion or fragment of anastellin (SEQ ID NO: 1) can incubated with fibronectin or fibrinogen to produce superfibronectin and superfibrinogen, respectively. Conversely, only a portion or fragment of fibronectin or fibrinogen can be incubated with anastellin (SEQ ID NO: 1) to produce superfibronectin or superfibrinogen, respectively. Similarly, a portion or fragment of anastellin (SEQ ID NO: 1) that retains functional activity with regard to inhibition of tumor growth, angiogenesis or metastasis is also encompassed by an anastellin useful in the compositions and methods of the invention. It is understood that the various constituent polypeptides and compositions can be attached to a polypeptide of the invention, for example, other polypeptides, carbohydrates, lipids, chemical moieties or polymerizing agents.

The constituent polypeptides of the compositions and methods of the invention, or any fibronectin or fibrinogen polymerizing agent that can generate superfibronectin or superfibrinogen, respectively, that retains at least one the functional activity described herein, can be isolated or synthesized using methods well known in the art. Such methods include recombinant DNA methods and chemical synthesis methods for production of proteins. For example, recombinant fibronectin and fibrinogen polypeptide fragments can be made in bacteria or chemically synthesized. Anastellin (SEQ ID NO: 1), fibronectin, fibronectin fragments, fibrinogen , fibrinogen fragments or any other constituent polypeptide of the invention can be isolated from animal tissue or plasma or produced and isolated from cell culture as well as from genetically altered animals, such as transgenic animals. Methods that can be used in synthesizing fibronectin or fibronectin fragments or modifications useful for generating superfibronectin are well known in the art, and include those described in Morla et al., supra, 1994.

The constituent polypeptides of the invention and fragments thereof can be purified by a variety of methods well-known in the art, including recombinant expression systems described herein, precipitation, gel filtration, ion-exchange, reverse-phase and affinity chromatography, and the like. Other well-known methods are described in Deutscher et al., “Guide to Protein Purification” Methods in Enzymology Vol. 182, (Academic Press, (1990)), which is incorporated herein by reference. Alternatively, the constituent polypeptides of the invention can be obtained using well-known recombinant methods as described, for example, in Sambrook et al., supra, (1989) and Ausubel et al., Current Protocols in Molecular Biology, John Wiley & Sons, New York (2000).

The methods and conditions for biochemical purification of a constituent polypeptide of the invention or fragment thereof can be chosen by those skilled in the art, and purification monitored, for example, by gel electrophoresis, an immunological assay, a binding assay, or a functional assay. For example, anastellin (SEQ ID NO: 1), fibronectin and fibrinogen as well as fragments of these polypeptides can be synthesized or obtained from plasma, cultured cells or any tissue source by methods well known in the art for protein isolation and purification. Constituent polypeptides of the invention and fragments thereof obtained from cultured cells can be natural or recombinant polypeptides. Furthermore, anastellin, fibronectin, superfibronectin and fibrinogen are commercially available from a variety of sources including, for example, Sigma Aldrich, St. Louis, Mo.

Methods for chemical and proteolytic cleavage and for purification of the resultant protein fragments are well known in the art (see, for example, Deutscher, Methods in Enzymology, Vol. 182, “Guide to Protein Purification,” San Diego: Academic Press, Inc. (1990), which is incorporated herein by reference). For example, a chemical such as cyanogen bromide or a protease such as trypsin, chymotrypsin, V8 protease, endoproteinase Lys-C, endoproteinase Arg-C or endoproteinase Asp-N can be used to produce fragments of the constituent polypeptides of the invention.

Methods for quantitative analysis of samples containing constituent polypeptides of compositions of the invention to determine the amount of a constituent polypeptide or composition of the invention are well known in the art and include absorption measurements in the ultraviolet and in the visibility range by direct or colorimetric protein determination. These methods are useful, for example, to determine the amount of superfibronectin or superfibrinogen formed upon incubation of fibronectin or fibrinogen, respectively, with a polymerizing agent such as anastellin (SEQ ID NO: 1). An appropriate method for protein quantification can be selected based on a variety of factors well known in the art, including protein purity and amount of sample. For protein samples consisting of different proteins quantitative detection of protein content can be achieved on the basis of the reactions shown by functional groups of the proteins to dye-forming reagents such that the intensity of the dye correlates directly with the concentration of the reacting groups and can be measured exactly.

Thus, the invention provides substantially pure compositions of anastellin (SEQ ID NO: 1) and fibrinogen as well as substantially pure compositions of superfibronectin and superfibrinogen. In addition, the invention provides a method of preparing superfibrinogen by contacting fibrinogen with anastellin (SEQ ID NO: 1) under conditions that allow formation of superfibrinogen. The invention also provides methods of inhibiting angiogenesis, metastasis and tumor growth by administering anastellin (SEQ ID NO: 1) or the compositions of the invention.

The invention further provides a method of inhibiting angiogenesis by administering anastellin (SEQ ID NO: 1) in an amount effective to inhibit angiogenesis, where the amount is 0.5 mg or greater, as well as a method of inhibiting tumor growth by administering anastellin (SEQ ID NO: 1) in an amount effective to inhibit angiogenesis, where the amount is 0.5 mg or greater. The invention also provides a method of inhibiting tumor growth by administering anastellin (SEQ ID NO: 1) in an amount effective to inhibit metastasis, where the amount is 0.5 mg or greater.

Additionally, the invention provides a method of inhibiting angiogenesis by administering fibrinogen, a fibrinogen polymerizing agent, fibronectin and a fibronectin polymerizing agent in an amount effective to inhibit angiogenesis. The invention also provides a method of inhibiting tumor growth by administering fibrinogen, a fibrinogen polymerizing agent, fibronectin and a fibronectin polymerizing agent in an amount effective to inhibit tumor growth. Also provided is a method of inhibiting metastasis by administering fibrinogen, a fibrinogen polymerizing agent, fibronectin and a fibronectin polymerizing agent in an amount effective to inhibit metastasis. Anastellin (SEQ ID NO: 1) and Factor XIIIa are fibrinogen polymerizing agents useful for practicing the methods of inhibiting angiogenesis, tumor growth and metastasis by administering fibrinogen (SEQ ID NO: 1) and a fibrinogen polymerizing agent in an amount effective to inhibit metastasis. Anastellin (SEQ ID NO: 1) is both, a fibrinogen and a fibronectin polymerizing agent and, as a result, particularly useful for practicing the methods of the invention. Anastellin can be mixed ex vivo with fibronectin or fibrinogen to polymerize these compounds, or can be administered to a subject and subsequently polymerize fibronectin and fibrinogen in vivo.

As used herein, the term “angiogenesis” means any growth of blood vessels or any neo-vascularization or re-vascularization of a tissue. The growth can, but must not necessarily, be stimulated by cytokines, such as cytokine-mediated activation of blood vessel endothelial cells.

As used herein, the terms “metastasis” and “metastases” refer to the movement of a tumor cell from its primary site by any means or by any route, including local invasion, lymphatic spread, vascular spread or transcoelomic spread.

As used herein, the term “effective amount” when used in reference to methods for inhibiting angiogenesis, is intended to mean any reduction in the growth of blood vessels or in the neo-vascularization or re-vascularization of a tissue when compared to treatment with an inactive control compound or absence of treatment. Furthermore, as used herein, the term “effective amount” in reference to methods for inhibiting tumor growth is intended to mean the amount of a composition or polypeptide of the invention that can reduce the number, size or proliferation of neoplastic cells when compared to treatment with an inactive control compound or absence of treatment. Similarly, when used in reference to methods for inhibiting metastasis, the term “effective amount” is intended to mean any reduction the movement of tumor cells from a primary site by any route, any decrease in the number of circulating tumor cells, any increase in the removal of tumor cells form the circulation or any reduction in the occurrence of neoplastic growth at secondary sites when compared to treatment with an inactive control compound or absence of treatment.

The actual amount considered to be an effective amount for a particular application can depend, for example, on such factors as the affinity, avidity, stability, bioavailability or selectivity of the molecule, the moiety attached to the molecule, the pharmaceutical carrier and the route of administration. Effective amounts can be determined or extrapolated using methods known to those skilled in the art. Such methods include, for example, in vitro assays with cultured cells or tissue biopsies and animal models known to those skilled in the art. For example, an appropriate amount and formulation for inhibiting tumor growth, metastasis or angiogenesis in humans can be extrapolated based on testing the efficacy of the compound in an animal model. By testing a spectrum of different dosage amounts, an optimum dosage can be determined and extrapolated for administration to a human subject.

The growth of solid tumors and the metastatic process is dependent on tumor angiogenesis. In humans, a tumor which is not able to stimulate its own vascularization can for years be restricted in growth to a microscopic region and limited to a million or less cells in size. Stimulation of blood vessel growth is a prerequisite of the conversion of a tumor to an angiogenic phenotype and involves a change in the local balance of blood vessel growth inhibitors and growth stimulators. In addition to allowing a tumor to increase in size, vascularization provides a means for tumor cell metastasis. The methods of the invention are useful in treating the types of cancer that are exhibit angiogenesis, solid tumor growth and metastasis. Tumor types that are susceptible to treatment with the methods provided by the invention include, for example, epithelial cancers such as breast cancer (Example II), melanomas, sarcomas (Example II), lymphomas and leukemias.

As shown in the Examples that follow, anastellin, superfibrinogen and superfibronectin can inhibit angiogenesis, tumor growth and metastasis. Since vascularization is a prerequisite for tumor growth, the anti-angiogenic effects of anastellin (SEQ ID NO: 1), superfibronectin and superfibrinogen, described in Example III, are likely related to the tumor inhibition effects of anastellin and the invention compositions, which are shown in Example II. Furthermore, the inhibition of angiogenesis, shown in Example III, with its suppressive effect on tumor growth, shown in Example II, can also underlie the anti-metastatic effect of anastellin (SEQ ID NO: 1), superfibronectin and superfibrinogen described in Example IV. As shown in Examples II to IV, the number of metastases correlated with the size of the primary tumor and the number of blood vessels in it. In particular, the ability to inhibit tumor growth of the compositions provided by the present invention correlates with a low blood vessel density in the tumors. Significantly, the blood vessel density in tumors following treatment with the invention compositions was only about twenty percent of that in control tumors. It is likely that the low number of blood vessels is an impediment to tumor growth, given that vascularization is a prerequisite for tumor growth as described in Hanahan and Folkman, Cell 86:353-364 (1996). The related decrease in metastasis (Table 1) is likely due to the reduced access of tumor cells to the circulation to establish metastatic colonies, a theory supported by earlier studies showing that superfibronectin inhibits lung colonization by tumor cells injected into the circulation (Pasqualini et al., supra, 1996).

The anti-metastatic activity of superfibronectin in experimental and spontaneous metastasis models has been described by Pasqualini et al., supra, 1996. The present invention provides methods for inhibiting angiogenesis, tumor growth and metastasis using anastellin (SEQ ID NO: 1) alone and further methods for inhibiting angiogenesis, tumor growth and metastasis using compositions of anastellin (SEQ ID NO: 1) and fibrinogen as well as compositions of superfibronectin and superfibrinogen.

The compositions of the invention can be formulated and administered by those skilled in the art in a manner and in an amount appropriate for the nature of the pathology to be treated; the weight, gender, age and health of the subject; the biochemical nature, bioactivity, bioavailability and side effects of the particular composition; and in a manner compatible with concurrent treatment regimens. For example, an appropriate amount and formulation for inhibiting tumor growth or angiogenesis in humans can be extrapolated from animal models known to those skilled in the art based on the particular disorder. It is understood, that the dosage of a composition administered to a subject has to be adjusted based on the bioactivity of the composition as well as on the metabolic characteristics of the subject. Therefore, once an optimum dosage has been determined based on testing a spectrum of different dosage amounts in an animal model, the optimum dosage amount can be extrapolated for administration to a human subject.

The compositions of the invention can be administered at various times based on the targeted results. It is understood that the timing for initiation of treatment can be determinative of the therapeutic results. In this regard, it is preferable to administer the compositions of the invention at an early stage of tumor growth so as to maximize the anti-angiogenic effects before large amounts of antagonistic angiogenic compounds are present. In addition, in order to prevent metastasis sustained administration of the invention compositions can take place over a prolonged time.

The total amount of a composition of the invention can be administered as a single dose or by infusion over a relatively short period of time, or can be administered in multiple doses administered over a more prolonged period of time. Such considerations will depend on a variety of factors such as, for example, the state of the disease and context of the treatment regimen. For example, if the goal is to inhibit metastasis or tumor growth the composition can be administered in a slow-release matrix, which can be implanted for systemic delivery or at the site of a desired target tissue. Contemplated matrices useful for controlled release of therapeutic compounds are well known in the art, and include materials such as DepoFoam™, biopolymers, micropumps, and the like. On the other hand, anastellin most effectively inhibits angiogenesis and tumor growth when administered in a single high dosage of 0.5 mg or greater. Based factors including, for example, tumor size and number of metastatic foci several doses of 0.5 mg can be administered at predetermined time intervals.

The compositions of the invention can be administered to the subject by any number of routes known in the art including, for example, systemically, such as intravenously or intraarterially. A composition of the invention can be provided in the form of isolated and substantially purified polypeptides and polypeptide fragments in pharmaceutically acceptable formulations using formulation methods known to those of ordinary skill in the art. These formulations can be administered by standard routes, including for example, topical, transdermal, intraperitoneal, intracranial, intracerebroventricular, intracerebral, intravaginal, intrauterine, oral, rectal or parenteral (e.g., intravenous, intraspinal, subcutaneous or intramuscular) routes. Preferred routes of administration that are particularly useful for administering the compositions of the invention include intraperitoneal and intravenous administration.

A composition of the invention can be administered as a solution or suspension together with a pharmaceutically acceptable medium. Such a pharmaceutically acceptable medium can be, for example, sterile aqueous solvents such as sodium phosphate buffer, phosphate buffered saline, normal saline or Ringer's solution or other physiologically buffered saline, or other solvent or vehicle such as a glycol, glycerol, an oil such as olive oil or an injectable organic ester. As described herein, superfibrinogen and superfibronectin can be prepared by mixing anastellin with fibrinogen and fibronectin, respectively, in a buffer that is appropriate for subsequent administration in vivo. A pharmaceutically acceptable medium can additionally contain physiologically acceptable compounds that act to, for example, stabilize the composition or increase its absorption. Such physiologically acceptable compounds include, for example, carbohydrates such as glucose, sucrose or dextrans; antioxidants such as ascorbic acid or glutathione; receptor mediated permeabilizers, which can be used to increase permeability of the blood-brain barrier; chelating agents such as EDTA, which disrupts microbial membranes; divalent metal ions such as calcium or magnesium; low molecular weight proteins; lipids or liposomes; or other stabilizers or excipients. Those skilled in the art understand that the choice of a pharmaceutically acceptable carrier depends on the route of administration of the compound containing the neutralizing agent and on its particular physical and chemical characteristics.

Formulations suitable for parenteral administration include aqueous and non-aqueous sterile injection solutions such as the pharmaceutically acceptable mediums described above. The solutions can additionally contain, for example, buffers, bacteriostats and solutes which render the formulation isotonic with the blood of the intended recipient. Other formulations include, for example, aqueous and non-aqueous sterile suspensions which can include suspending agents and thickening agents. The formulations can be presented in unit-dose or multi-dose containers, for example, sealed ampules and vials, and can be stored in a lyophilized condition requiring, for example, the addition of the sterile liquid carrier, immediately prior to use. Extemporaneous injection solutions and suspensions can be prepared from sterile powders, granules and tablets of the kind previously described.

A constituent polypeptide or composition of the invention can be incorporated into a material that allows for sustained release of the composition useful for inhibiting tumor growth, angiogenesis or metastasis. The sustained release form has the advantage of inhibiting growth, metastases, endothelial growth or the like over an extended period of time without the need for repeated administrations. Sustained release can be achieved, for example, with a sustained release material such as a wafer, an immunobead, a micropump or other material that provides for controlled slow release. Such controlled release materials are well known in the art and available from commercial sources (Alza Corp., Palo Alto Calif.; Depotech, La Jolla Calif.; see, also, Pardoll, Ann. Rev. Immunol. 13:399-415 (1995), which is incorporated herein by reference). In addition, biodegradable polymers and their use are described, for example, in Brem et al., J. Neurosurg. 74:441-446 (1991), which is incorporated herein by reference. In addition, a bioerodible or biodegradable material that can be formulated with anastellin (SEQ ID NO: 1) or any of the compositions of the invention, such as polylactic acid, polygalactic acid, regenerated collagen, multilamellar liposomes or other conventional depot formulations, can be implanted to slowly release anastellin (SEQ ID NO: 1) or a particular composition of the invention. The use of infusion pumps, matrix entrapment systems, and transdermal delivery devices also are contemplated in the present invention.

Anastellin (SEQ ID NO: 1) and the compositions of the invention also can be advantageously enclosed in micelles or liposomes. Liposome encapsulation technology is well known. Liposomes, which consist of phospholipids or other lipids, are nontoxic, physiologically acceptable and metabolizable carriers that are relatively simple to make and administer, can be targeted to a specific tissue, such as neural tissue, through the use of receptors, ligands or antibodies capable of binding the targeted tissue. The technology and preparation of such formulations is well known in the art, see, for example, Pardridge, supra, 1991; Radin, et al., Meth. Enzymol. 98:613-618 (1983); Gregoriadis, Liposome Technology, Vols. I to III, 2nd ed. (CRC Press, Boca Raton Fla. (1993)) and Nabel, G. J., et al., Proc. Natl. Acad. Sci. USA 90:11307-11311 (1993), which are incorporated herein by reference. It is understood that liposomes are desirable for applications that require an increase in the lipophilicity of the compound such as those applications that involve crossing of the blood-brain barrier.

The invention also provides for methods in which anastellin (SEQ ID NO: 1), superfibronectin or superfibrinogen are generated in vivo. These methods include implanting into the subject a cell genetically modified to express and secrete anastellin (SEQ ID NO: 1) or any of the constituent polypeptides in vivo. The invention methods also encompass gene therapy involving inserting into the subject genes that are capable of expressing anastellin (SEQ ID NO: 1) or any of the constituent polypeptides in vivo. For a subject suffering from a long-term risk of metastasis or tumor recurrence, such methods have the advantage of obviating or reducing the need for repeated administration.

For ex vivo gene transfer, using methods well known in the art, a cell can be transiently or stably transfected with an expression vector containing the desired nucleic acid sequences, for example as described in Chang, Somatic Gene Therapy, CRC Press, Boca Raton (1995), which is incorporated herein by reference. The transfected cell is then implanted into the subject. Methods of transfecting cells ex vivo are well known in the art, for example, Kriegler, Gene Transfer and Expression: A Laboratory Manual, W. H. Freeman & Co., New York (1990), incorporated herein by reference. For the transfection of a cell that continues to divide such as a fibroblast, muscle cell, glial cell or neuronal precursor cell, retroviral or adenoviral vectors can be used. For the transfection of an nucleic acid into a postmitotic cell such as a neuron, for example, a replication defective herpes simplex virus type 1 or Sindbis virus vector can be used, and such methods are well known in the art, as in During, et al., Soc. Neurosci. Abstr. 17:140 (1991); Sable, et al., Soc. Neurosci. Abstr. 17:570 (1991); Dubensky, T. W., et al., J. of Virology 70:508-519 (1996), each of which is incorporated herein by reference.

For in vivo gene therapy, using methods well known in the art, the desired cell or tissue can be transiently or stably transfected with an expression vector containing the desired nucleic acid sequence(s) to effect expression of anastellin (SEQ ID NO: 1) or any of the constituent polypeptides of the invention in vivo, for example, as described in Acsadi, G., et al., New Biol. 3:71-81 (1991); Chang, supra; Chen, S., et al., Proc. Natl., Acad. Sci. USA 91:3054-3057 (1994); Culver, K. W., et al., Science 256:1550-1552 (1992); Furth, P. A., et al., Molec. Biotech. 4:121-127 (1995); which are incorporated herein by reference.

In current cancer treatment regimes, more than one compound is often administered to an individual for management of the same or different aspects of the disease. Thus, for use in inhibiting tumor growth. metastasis or angiogenesis, a composition of the invention can advantageously be formulated with a second compound such as a antineoplastic agent such as, for example, tamoxifen, doxorubicin or cyclophosphamide, as well as with compounds administered to reduce side-effects of antineoplastic agents. Contemplated methods of inhibiting tumor growth, metastasis and angiogenesis include administering a compound of the invention alone, in combination with, or in sequence with, such other compounds. Alternatively, combination therapies can consist of fusion proteins, where a constituent polypeptide of a composition of the invention is linked to a heterologous protein, such as a therapeutic protein or targeting protein. Heterologous proteins useful for practicing this embodiment of the invention include, for example, RGD peptides, endostatin and angiostatin. The compositions of the invention can be administered as part of a treatment regimen that includes, for example, radiation, chemotherapy, antibody therapy or any combination of these and other therapies.

It is understood that modifications which do not substantially affect the activity of the various embodiments of this invention are also included within the definition of the invention provided herein. Accordingly, the following examples are intended to illustrate but not limit the present invention.

It is understood that modifications which do not substantially affect the activity of the various embodiments of this invention are also included within the definition of the invention provided herein. Accordingly, the following examples are intended to illustrate but not limit the present invention.

### EXAMPLE I

### Anastellin Induces Formation of Superfibrinogen

This example describes the addition of anastellin (SEQ ID NO: 1) to a fibrinogen solution in vitro, which caused turbidity and produced a polymeric compound termed superfibrinogen.

Anastellin (SEQ ID NO: 1)and III₁₁-C, a control fibronectin fragment from type III repeat 11, were prepared as recombinant his-tagged proteins in bacteria and purified as described in Morla el al., supra, 1994, and Pasqualini et al., supra, 1996. Human plasma fibronectin was obtained from Chemicon (Temecula, Calif.) and human fibrinogen was obtained from Sigma (St.Louis, Mo.). Fibronectin was converted to superfibronectin by mixing 100 μg fibronectin in 100 μl PBS with 300 μg anastellin (SEQ ID NO: 1) in 100 μl PBS as described in Pasqualini et al., supra, 1996. Anastellin (SEQ ID NO: 1) also binds to fibrinogen (Morla and Ruoslahti, 1992), and substitution of fibrinogen (from human plasma, fraction I, Sigma product #F4883) for fibronectin yielded polymerized fibrinogen (“superfibrinogen”; sFBG) as described below. Protein solutions were sterilized by filtering through 0.2 μm membrane prior to polymerization.

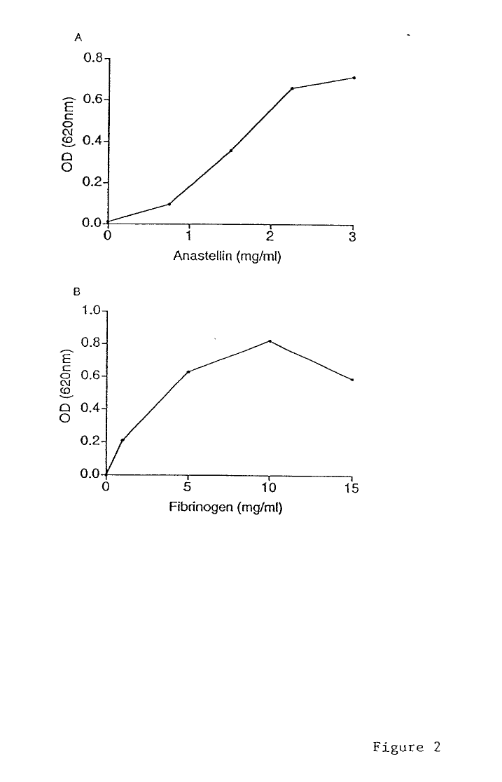

Equal volumes of anastellin (SEQ ID NO: 1) and fibrinogen at various concentrations were mixed and protein polymerization was monitored by measuring optical density at 620 nm (turbidity). FIG. 2A shows the increase in turbidity observed when a constant amount of fibrinogen (5 mg/ml) was mixed with increasing amounts of anastellin (SEQ ID NO: 1). Fidure 2B shows the change in turbidity observed when a constant amount of anastellin (3 mg/ml) was mixed with an increasing amount of fibrinogen. Mixing fibrinogen with a control fragment from the 11th type III repeat of fibronectin (III₁₁-C; 3 mg/ml) yielded essentially baseline turbidity.

### EXAMPLE II

### Anastellin, Superfibronectin and Superfibrinogen Inhibit Tumor Growth

This example describes the anti-tumor effects of systemically administered superfibronectin, superfibrinogen and anastellin.

The C8161 melanoma, KRIB osteosarcoma, and MDA-MB-435 breast carcinoma human tumor cell lines were cultured and harvested used to establish human xenograft tumors in nude mice as described in Pasqualini et al., supra, 1996 and Arap et al., Science 279: 377-380 (1998), which are incorporated herein by reference. Briefly, the cells were allowed to grow in the continuous culture for no more than three consecutive passages before being used in the experiments. Actively growing cells were detached from culture plates with PBS/2.5 mM EDTA or Trypsin-EDTA (0.25% trypsin, 1 mM Na-EDTA; Gibco BRL, Rockville, Md.). The detached cells were resuspended in DMEM, counted and examined for viability by trypan blue exclusion. Subsequently, the cells were injected into mice as described below. A portion of the cells used in the injections was seeded back into a culture plate to determine plating efficiency. The viability was higher than 99% and the plating efficiency greater than 95%.

The tumor cells were injected into two-month old immunodeficient Balb/c/nu/nu, female mice (Harlan Sprague-Dawley, San Diego, Calif.). Briefly, to obtain subcutaneous tumors, 10⁶ tumor cells suspended in 200 μl of DMEM were injected into the right posterior flank of the mice, which were randomized and divided into experimental groups of 5-6 mice per group. At 3 weeks after tumor cell implantation nearly all of the mice had developed palpable tumors. The mice were treated with intraperitoneal injections of either anastellin, superfibronectin or superfibrinogen in PBS, or with PBS alone. The treatments were administered twice a week via intraperitoneal injections for the duration of the study. The injections were given in 200 μl of PBS. In some experiments unpolymerized fibronectin, unpolymerized fibrinogen or the III₁₁-C fragment of fibronectin were used as additional controls.

To monitor tumor growth during the treatment, tumor size was estimated by taking biweekly measurements of the longest and shortest diameter and averaging the two measurements. At about 8 weeks after tumor cell implantation, which corresponds to about 5 weeks after the start of the treatments, the mice were anesthetized and perfused through the heart with PBS.

### Inhibition of Human Xenograft Tumors from C8161 Cells

Subcutaneous tumors were grown in nude mice form C8161 melanoma cells as described above. Treatment with biweekly intraperitoneal injections of fibronectin, fibronectin mixed with anastellin, or anastellin alone was started three weeks after the tumor implantation and continued for 3 weeks. The injections consisted of vehicle, 100 μg of fibronectin, 100 μg of fibronectin mixed with 300 μg of anastellin (sFN), or 300 μg of anastellin alone. The increase in size of the tumors during the experiment is shown in FIG. 1. The tumors of the mice treated with superfibronectin or anastellin grew significantly less than the tumors of the mice treated with vehicle alone (p<0.001 and p<0.05, respectively).

### Inhibition of KRIB Osteosarcoma Growth

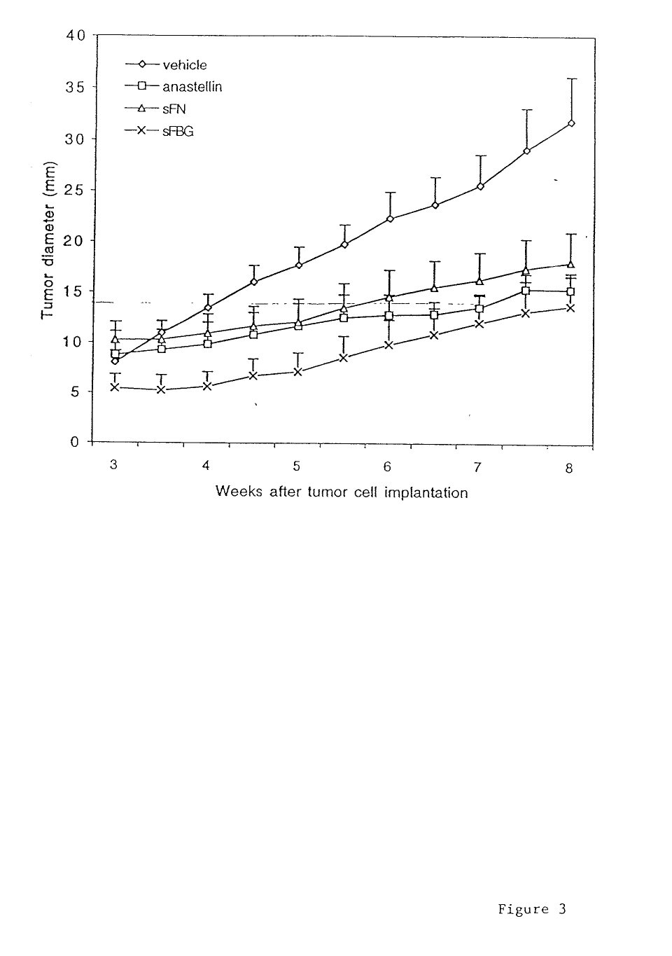

Subcutaneous tumors were grown in nude mice from KRIB osteosarcoma cells cutured and harvested as described above. Treatments with biweekly intraperitoneal injections of 6 mice per treatment group of either superfibronectin, superfibrinogen or anastellin were started three weeks after tumor implantation and continued for 5 weeks. Each injection consisted of 100 μg of fibronectin mixed with 300 μg of anastellin (sFN), 500 μg of fibrinogen mixed with 300 μg of anastellin (sFBG), or 600 μg of anastellin. FIG. 3 shows tumor sizes (mean and SEM) in each treatment group at the indicated time point. The tumors of the mice treated with anastellin, superfibronectin and superfibrinogen were significantly smaller at the end of the experiment than those of the mice treated with vehicle alone (anastellin, p<0.01; sFN, p<0.05; sFBG p<0.01).

### Inhibition of MDA-MD-435 Breast Carcinoma Growth

Nude mice bearing subcutaneous tumors from MDA-MB-435 breast carcinoma cells were used in a treatment experiment as described above for KRIB osteosarcoma. Each injection consisted of 100 μg of fibronectin mixed with 300 μg of anastellin (sFN), 500 μg of fibrinogen mixed with 300 μg of anastellin (sFBG), 600 μg of anastellin or 600 μg of III₁₁-C and 6 mice were used per group. The tumors were significantly smaller at the end of the study in the mice treated with anastellin (p<0.01), superfibronectin (p<0.001) or sFBG (p<0.001) than in the mice treated with either vehicle alone or with III₁₁-C.

The doses per injection were: anastellin, 600 μg; superfibronectin, 100 μg of fibronectin mixed with 300 μg of anastellin; sFBG, 500 μg of fibrinogen mixed with 300 μg of anastellin; III₁₁-llC, 600 μg, and 6 mice were used per group. The tumors were significantly smaller at the end of the experiment in the mice treated with anastellin (p<0.01), superfibronectin (p<0.001) or sFBG (p<0.001) than in the mice treated with vehicle alone, or with III₁₁-C.

### Relative Inhibition of Tumor Growth by Anastellin, Superfibronectin and Superfibrinogen

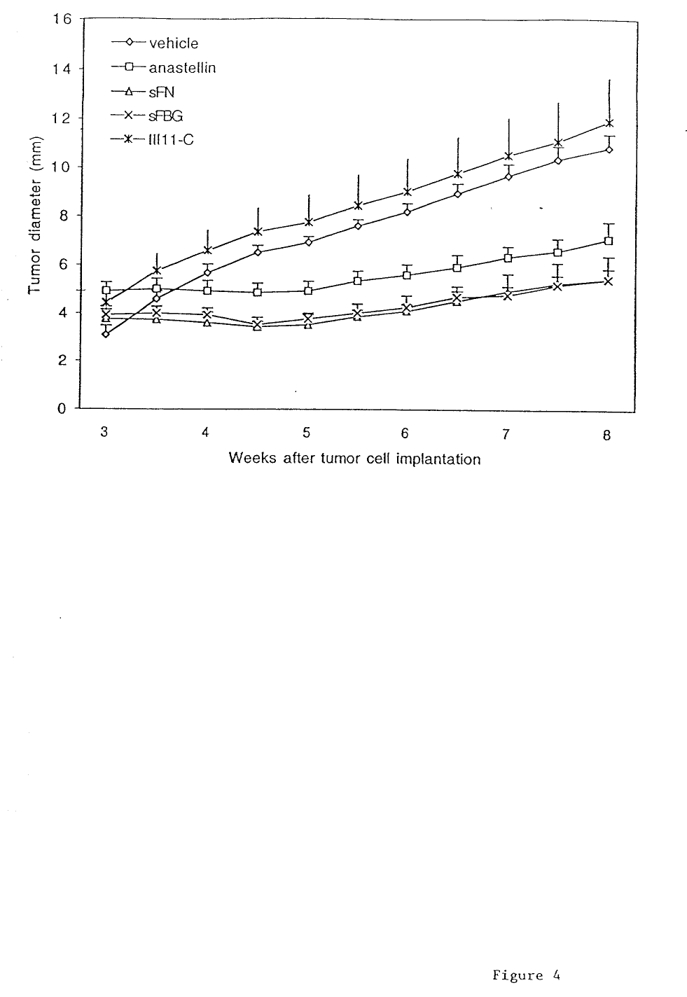

Anastellin alone inhibited tumor growth approximately as effectively as superfibronectin or superfibrinogen. The results obtained were similar for C8161, KRIB human osteosarcoma (FIG. 3) and MDA-MB-435 human breast carcinoma (FIG. 4). A peptide analogous to anastellin but derived from the 11th type III repeat of fibronectin (III₁₁-C)) was used as an additional control in some experiments. This peptide does not bind to fibronectin (Morla et al., supra, 1994) and had no effect on the growth of the breast carcinoma tumors. In some experiments, we also included as controls non-polymerized fibronectin and fibrinogen at the same dose as that used in the polymers. These controls had no effect on tumor growth (FIG. 1).

### EXAMPLE III

### Anastellin, Superfibronectin and Superfibrinogen Inhibit Tumor Angiogenesis

This example describes inhibition of tumor angiogenesis by anastellin, superfibronectin and superfibrinogen.

To determine whether the inhibition of tumor growth is due to the inhibition of tumor angiogenesis the blood vessel density was determined using sections of tumors collected at the termination of the tumor growth inhibition studies described above. Paraffin embedding, sectioning and immunostaining for blood vessels with anti-CD31 (rat anti-mouse, Pharmingen, San Diego, Calif.) were carried out in The Burnham Institute Histology Facility.

### Reduced Tumor Angiogenesis in KRIB Osteosarcoma Tumors

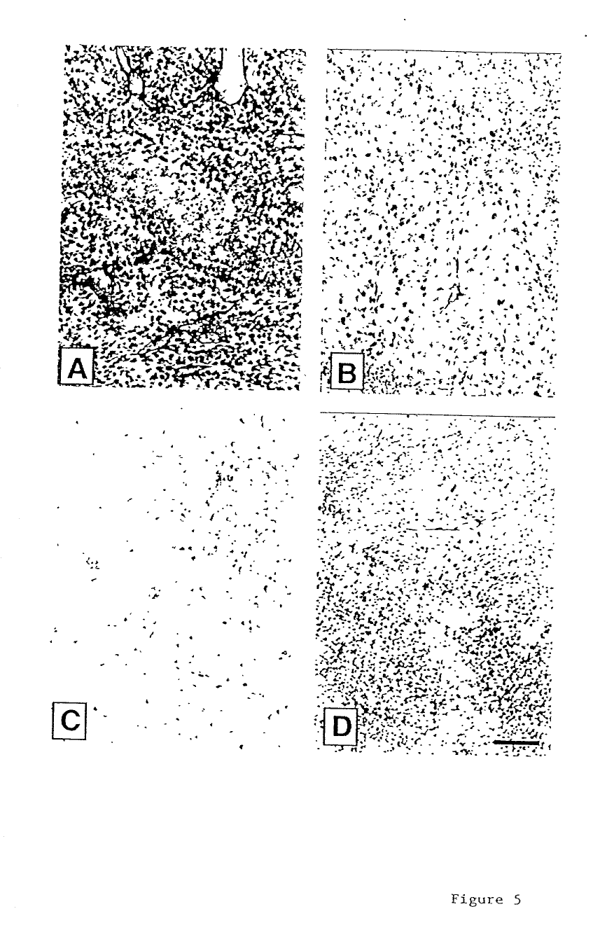

KRIB osteosarcoma tumors from a tumor growth inhibition study similar to the one described above and shown in FIG. 3 were removed at the termination of the study, sectioned, and the sections were stained with anti-CD31 antibodies to visualize tumor blood vessels. Representative microscopic fields from the tumors showed higher density of blood vessels in the vehicle alone group (FIG. 5A) than in the anastellin (FIG. 5B), superfibronectin (FIG. 5C), and superfibrinogen (FIG. 5D) groups.

### Reduced Tumor Angiogenesis in MDA-MB-435

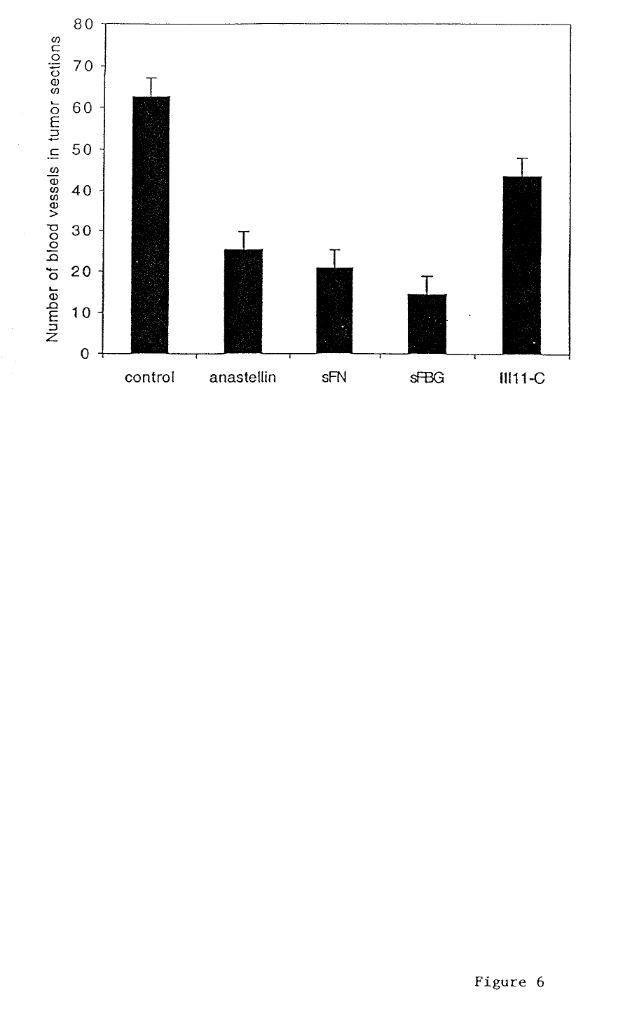

MDA-MB-435 tumors from mice treated with anastellin, superfibronectin or sFBG. Blood vessels in tumor sections stained with anti-CD31 were counted from 5 microscopic fields for all tumors in the experiment described above and shown in FIG. 4. FIG. 6 shows the mean and SEM for the number of blood vessels in the tumors from the six mice in each of the MDA-MB-435 treatment groups. The blood vessel density was significantly reduced in each of the anastellin (p<0.02), superfibronectin (p<0.02) and superfibrinogen (p<0.01) groups relative to the vehicle alone group and the other control group that received III₁₁-C.

### Relative Inhibition of Tumor Angiogenesis by Anastellin, Superfibronectin and Superfibrinogen

Staining for the endothelial marker, CD31, showed greatly reduced blood vessel density in the tumors of the mice treated with anastellin, superfibronectin and superfibrinogen relative to the vehicle-treated controls. The results were similar for all of the three tumor types, human xenograft tumors from C8161 cells, KRIB osteosarcoma cells and MDA-MB-435 cells. In contrast to the tumor growth inhibition results for these tumors, there were no discernible differences in the density of CD31-positive vessels in the lungs of the mice that received the three treatments, anastellin, superfibronectin and superfibrinogen. These results suggest that the inhibition of tumor growth by anastellin, superfibronectin and superfibrinogen is, at least in part, due to suppression of tumor angiogenesis.

### EXAMPLE IV

### Anastellin, Superfibronectin and Superfibrinogen Inhibit Metastasis

This example describes inhibition of metastasis by anastellin, superfibronectin and superfibrinogen.

At the termination of the tumor growth inhibition experiments or, if earlier, at death, tumors and lungs were excised, weighed and the tissues were fixed in 4 percent paraformaldehyde for 24 hours. The tissues were then stored in 70 percent ethanol and subsequently examined for the number of metastatic foci. Macroscopic tumor foci on the surface of the lungs were noted and microscopic lesions were counted in tissue sections.

Table 1 shows the number of mice with lung metastases among mice bearing C8161 or KRIB tumors and treated with various anastellin compounds. The KRIB and C8161 tumor mice treated with vehicle alone invariably showed evidence of lung metastasis; the MDA-MB-435 tumors did not metastasize within the timeframe of the study. As shown in Table 1, fewer KRIB or C8161 tumor mice developed metastatic foci in the lungs when treated with superfibronectin than did mice in the control groups. The metastasis inhibition was significant for both tumors in the mice treated with anastellin and superfibronectin, while the anti-metastatic effect of superfibrinogen seemed somewhat weaker.

TABLE 1
Anti-Metastatic Effects of Anastellin Compounds.

| | Vehicle | Anastellin | sFN | sFBG |
|---|---|---|---|---|
| Tumor | | | | |
| C8161 | 17/17 | 4/11** | 1/11*** | — |
| KRIB | 9/12 | 3/12* | 4/12* | 5/12† |

In Table 1 * indicates P<0.05, ** indicates P<0.01, and *** indicates P<0.001 relative to the vehicle group. In addition, [symbol] denotes the sFBG group, which was not significantly different from the control group (P=0.11) although the number of metastatic foci (not shown) was significantly smaller than in the controls (P<0.01).

Significantly, the anti-metastatic effect of superfibronectin can be reproduced with anastellin (SEQ ID NO: 1) alone. In addition, anastellin (SEQ ID NO: 1) is anti-angiogenic and suppresses tumor growth. Overall, the number of mice with metastatic foci was lower in the anastellin, superfibronectin and superfibrinogen treatment groups than in the control groups.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

What is claimed is:

1. A substantially pure composition comprising anastellin (SEQ ID NO: 1) and fibrinogen.

2. A substantially pure composition comprising superfibrinogen (sFBG) and superfibronectin (sFN).

3. A method of preparing superfibrinogen (sFBG) comprising contacting fibrinogen with anastellin (SEQ ID NO: 1) under conditions that allow formation of super-fibrinogen (sFBG).

4. A method of inhibiting angiogenesis comprising administering anastellin (SEQ ID NO: 1) in an amount effective to inhibit angiogenesis, wherein said amount is 0.5 mg or greater.

5. A method of inhibiting tumor growth comprising administering anastellin (SEQ ID NO: 1) in an amount effective to inhibit tumor growth, wherein said amount is 0.5 mg or greater.

6. A method of inhibiting angiogenesis comprising administering fibrinogen and a fibrinogen polymerizing agent in an amount effective to inhibit angiogenesis.

7. The method of claim 6, wherein said fibrinogen polymerizing agent is Factor XIIIa.

8. The method of claim 6, wherein said fibrinogen polymerizing agent is anastellin (SEQ ID NO: 1).

9. A method of inhibiting tumor growth comprising administering fibrinogen and a fibrinogen polymerizing agent in an amount effective to inhibit tumor growth.

10. The method of claim 9, wherein said fibrinogen polymerizing agent is Factor XIIIa.

11. The method of claim 9, wherein said fibrinogen polymerizing agent is anastellin (SEQ ID NO: 1).

12. A method of inhibiting metastasis comprising administering fibrinogen and a fibrinogen polymerizing agent in an amount effective to inhibit metastasis.

13. The method of claim 12, wherein said fibrinogen polymerizing agent is Factor XIIIa.

14. The method of claim 12, wherein said fibrinogen polymerizing agent is anastellin (SEQ ID NO: 1).
